(19) United States
(12) Patent Application Publication
(10) Pub. No.: US 2010/0298680 A1
(43) Pub. Date: Nov. 25, 2010

(54) METHOD AND DEVICE FOR DETERMINING A PROPERTY OF LIVING TISSUE

(76) Inventors: Mark Stuart Talary, Zurich (CH); Francois Dewarrat, Zurich (CH); Daniel Huber, Zurich (CH); Lisa Falco, Zurich (CH)

Correspondence Address: LADAS & PARRY LLP, 26 WEST 61ST STREET, NEW YORK, NY, 10023, US

(21) Appl. No.: 12/809,099

(22) PCT Filed: Jan. 11, 2008
(86) PCT No.: PCT/CH08/00016

§ 371 Date: Aug. 3, 2010

(51) Int. Cl. A61B 5/1477; A61B 5/053

(52) U.S. Cl. 600/347; 600/547

(57) ABSTRACT

The invention relates to a measurement of tissue properties, in particular glucose, by measuring the response of the tissue to an applied electric field. The tissue is modeled by a System of homogeneous layers. In one approach, a plurality of electrical fields are generated in the tissue at different frequencies. For each of the fields, a signal depending on the dielectric permittivity as seen by the electrode arrangement at the frequency is measured, thereby generating a measured dataset. In another approach the different electrode configurations can be used to achieve different penetration depths in the desired layers. A function is then fitted to the dataset by varying at least some parameters of the function. These parameters describe the dispersion of the dielectric permittivity of a plurality of layers in the tissue. At least part of the parameters obtained in this fitting procedure are then used for determining the desired tissue property. Furthermore a combination of these two approaches can be used to optimize the uniqueness of the Solution of the fitting procedure for changes at a specific depth.

### TECHNICAL FIELD

The invention relates to a method and a device for determining a property of living tissue, in particular but not exclusively for the purpose of measuring the glucose level in the tissue.

### BACKGROUND ART

WO 02/069791 describes a device for measuring blood glucose in living tissue. It comprises an electrode arrangement with a ground electrode and a signal electrode. A signal source applies an electrical AC-signal of known voltage or current through a resistor to the electrodes, and a detector determines the voltage over or current through the electrodes. This voltage or current depends on the dielectric properties of the tissue, measured as an impedance or admittance which, as it has been found, are indicative of the glucose level within the tissue.

WO 2005/120332 describes another embodiment of such a device where a plurality of electrical fields are generated by applying voltages to different configurations of the electrode arrangement, thereby generating fields of different spatial configurations within the tissue. This allows, for example, a reduction of the influence of surface effects on the measured signal.

These techniques allow to measure a property c of living tissue, in particular the glucose level, which property c affects the complex dielectric permittivity ∈(ω) of the tissue. They rely on applying an electrode arrangement to a skin region of the tissue and generating electrical fields within the tissue. For each field, a signal depending on the bulk dielectric properties as seen by the electrode arrangement is measured. The measured signal is then processed, e.g. using pre-recorded calibration data, in order to obtain the desired property c.

### DISCLOSURE OF THE INVENTION

The object of the present invention is to provide a device of this type that further improves the accuracy of the measured signal.

This object is achieved by the method and device according to the independent claims.

Hence, in a first aspect of the invention, a plurality of electrical fields are generated in the tissue at different frequencies ω_{w} with w=1 to W, with W >1. For each one of the fields, a signal s_{w} with w=1 to W, depending on the dielectric permittivity ∈(ω_{w}) as seen by the electrode arrangement at the frequency ω_{w}, is measured, thereby generating a measured dataset. A function F1 is then fitted to the dataset by varying at least some parameters of the function F1. The parameters of the function, called p₁₁, . . . , p_{MN}, describe the effective dispersion of the dielectric properties of a plurality of individual layers in the tissue. At least part of the parameters obtained in this fitting procedure are then used for determining the desired tissue property c.

This technique therefore relies on a model where the tissue is considered to consist of several layers m=1 . . . M. Each layer m may have a different dispersion of the complex dielectric permittivity, expressed by the dispersion parameters pₘ₁ . . . p_{mN}.

It has been found that the dispersion parameters of some of these layers, e.g. of the dermis layer, depend more strongly on the desired property c, e.g. glucose concentration, than others, e.g. the stratum corneum or the epidermis.

This procedure improves the measurement accuracy for various reasons. On the one hand, by considering the tissue to be built up of separate layers, with each layer having it own dielectric properties, a more accurate model of the tissue is created. Furthermore, the measurement at several frequencies in combination with a model of layers where each layer exhibits its own, specific dispersion, allows to perform a depth-resolved measurement that yields the dispersion parameters of the different layers. Finally, since the procedure allows to distinguish between the parameters of the different layers, the analysis can focus on the parameters of those layers that are most strongly influenced by the desired property c.

A second aspect of the invention is also based on a multi-layer model of the tissue and on applying different electrical fields thereto. In this aspect, however, the fields may have the same frequency but they differ in spatial distribution. For this purpose, voltages are applied to different configurations u of the electrode arrangement, with u=1 to U and U>1. For each configuration u, a signal sᵤ is measured, where the signal depends on the bulk effective complex dielectric permittivity sᵤ as seen by the electrode arrangement for configuration u. The dataset measured in this way is used to find an at least approximate solution for a set of equations of the type

sᵤ=F0_{u(∈}₁, . . . ∈_{M},d₁, . . . d_{M-1}),

with u=1 to U, by varying at least part of the parameters ∈₁, . . . ∈ₘ, d₁, . . . d_{M-1}. The signal s can be the complex impedance (described with a phase and amplitude) or the admittance (described with a complex capacitance). These parameters describe the complex dielectric permittivity and thickness of each layer. At least part of the values of the varied parameters obtained in this way are then used for calculating the property c.

Hence, in this second aspect, spatially different fields are applied to the tissue, each of which affects the different layers differently, which again allows to determine the parameters of the individual layers by minimizing the errors in the set of equations mentioned above.

Again, this method allows to focus the analysis on the parameters of the layer most sensitive to the desired property c.

The invention also relates to a device comprising a control unit adapted to carry out the steps of the above methods.

The invention is especially suited for determining glucose, albeit it can also be used for determining other tissue properties, such as an electrolyte level.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 is a cross section of a device for measuring a glucose level,

FIG. 2 is a block circuit diagram of the device of FIG. 1,

FIG. 3 is an illustration of the layer model of the tissue (not to scale),

FIG. 4 shows a comparison between simulations and measurements, the change in signals with electrode geometries and the effect of penetration depth of the electric field.

FIG. 5 shows example glucose data estimated by a non-lossy model compared to data obtained in reference measurements,

FIG. 6 shows example glucose data estimated by a lossy model compared to data obtained in reference measurements, and

FIG. 7 shows example glucose data estimated by a refined lossy model compared to data obtained in reference measurements.

### MODES FOR CARRYING OUT THE INVENTION

### Device Setup

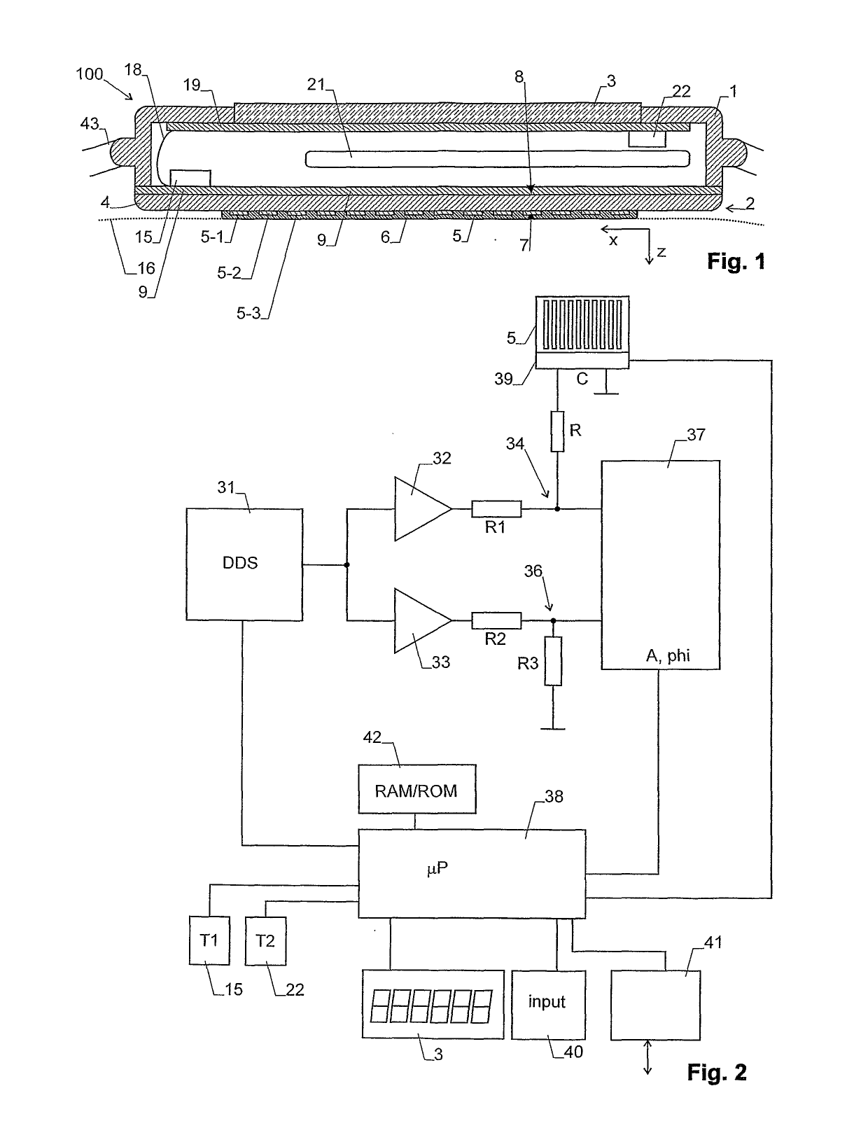

FIG. 1 shows a cross section of an embodiment of a device 100 for measuring a patient's glucose level or some other parameter c in a patient's body, such as an electrolyte level of the tissue. It comprises a housing 1 closed on one side by an electrode plate 2. A display 3 is arranged opposite electrode plate 2. Electronic circuitry is arranged between electrode plate 2 and display 3. Alternatively, at least part of the circuitry and/or the display can be located in an external device that communicates with device 100 by means of wireless or wire-bound communication.

Electrode plate 2 comprises an electrically insulating substrate 4. An electrode arrangement 5 comprising e.g. a plurality of parallel strip electrodes 5-0, 5-1, 5-2, etc. or concentric ring electrodes and optionally being covered by an insulating layer 6 may be arranged on an outer side 7 of insulating substrate 4. An inner side 8 of the insulating substrate 4 may be covered by a ground electrode 9. Suitable through-contacts (not shown) connect the strip electrodes 5-i to contact pads arranged on inner side 8.

Advantageously, a first temperature sensor 15 is mounted to ground electrode 9 in direct thermal contact thereto and measures a first temperature Ti.

Leads or springs 18 are provided to connect ground electrode 9, the contact pads and first temperature sensor 15 to the electronic circuitry arranged on a printed circuit board 19 forming an assembly of electronic components. A battery 21 for powering the circuitry is arranged between printed circuit board 19 and electrode plate 2. A second temperature sensor 22 can be arranged on printed circuit board 19 and in direct thermal contact thereto for measuring a second temperature T2.

FIG. 2 shows a block circuit diagram of the circuitry of device 100. It comprises a voltage generated by direct digital synthesis (DDS) to produce a controllable signal oscillation 31 as a signal source for generating a sine wave signal or another periodic signal. Instead of an oscillator, a pulse generator could be used for generating substantially non-periodic signals, such as short pulses or step-like voltage transitions. The signal from the signal source is fed to two amplifiers 32, 33. The output of first amplifier 32 is connected via a resistor R1 to a first signal path 34. A resistive R and the capacitive load of the electrode arrangement 5 are connected in series between first signal path 34 and ground. A switching assembly 39 can be provided to selectively connect the electrodes 5-i to either resistor R or ground, thereby defining at least two different electrode configurations that allow to apply different voltage patterns to the surface of the tissue. An embodiment of the switching assembly is described in WO 2005/120332, the disclosure of which is incorporated by reference herein.

The output of second amplifier 33 is connected via a resistor R2 to a second signal path 36. Second signal path 36 can be substantially identical to first signal path 34 but comprises a resistor R3 as a reference load.

Both signal paths 34, 36 are fed to a measuring circuit 37, which determines the relative amplitude A of both signals and/or their mutual phase shift φ, deriving therefrom at least one measured signal s. Relative amplitude A can e.g. be the amplitude of first signal path 34 in units of the amplitude of second signal path 36 (wherein the amplitudes are the peak values of the sine waves or, if pulses or voltage steps are used as measuring signal, the corresponding peak amplitude or step voltage).

The output signal of measuring circuit 37 is fed to a microprocessor 38, which also controls the operation of DDS 31.

Microprocessor 38 further samples the first and second temperature signals T1, T2 from first and second temperature sensors 15, 22. It also controls display device 3, an input device 40 with user operable controls, and an interface 41 to an external computer. A memory 42 is provided for storing calibration parameters, measurement results, further data processing as well as firmware for microprocessor 38. At least part of memory 42 is non-volatile.

The electrodes of electrode arrangement 5 are arranged on the skin 16 of the patient as shown in FIG. 1. For a good and permanent contact with the patient's skin, the device is advantageously worn on an arm or leg and provided with a suitable holder or flexible band attachment 43.

In summary, the device shown in FIGS. 1 and 2 comprises:
a control unit formed by microprocessor 38 and its peripheral components,
an electrode arrangement 5,
a signal source (DDS 31) for applying an electrical signal to electrode arrangement 5 for generating an electrical field in the tissue, and
a detector for measuring a response from the tissue to the electrical field and for determining at least one parameter therefrom, the detector primarily comprising the elements 37, 38.

Model:

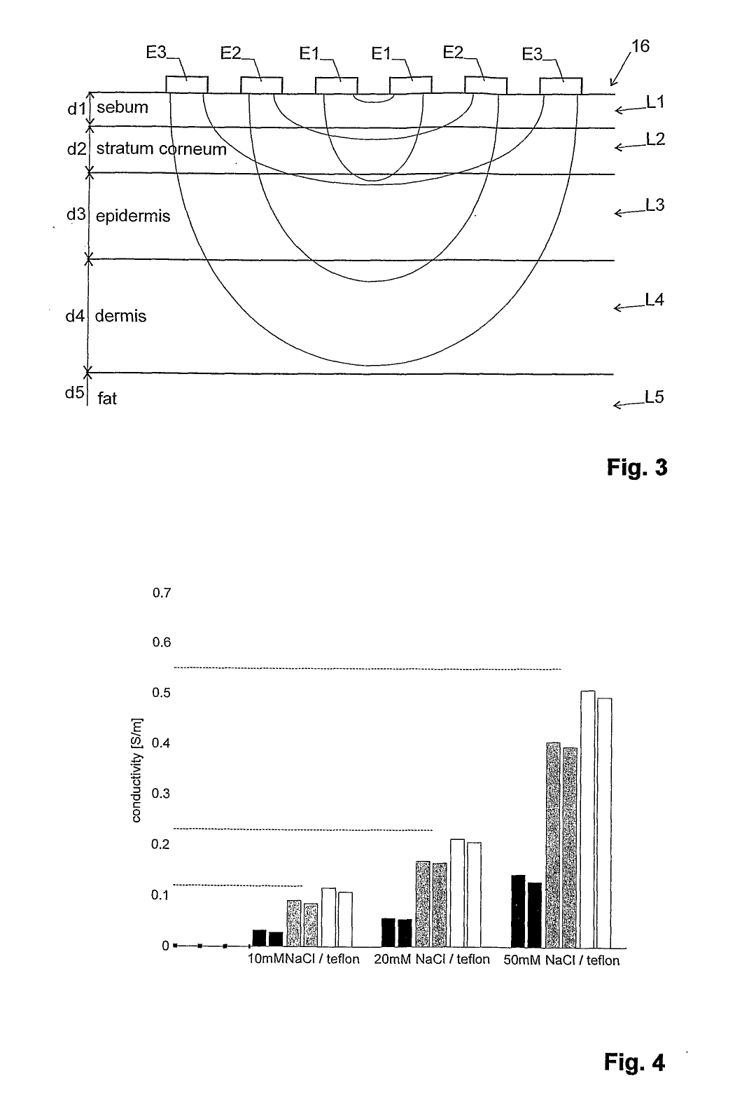

Before providing a more detailed description of the operation of the device, a model of the tissue to be measured is described by reference to FIG. 3. As shown therein, the tissue is assumed to consist of several layers L1, L2, L3, LM namely a total of M>1 layers. The layers are characterized by their respective thicknesses d₁, . . . , d_{M} and their complex dielectric permittivity

$${ɛ}_{m}^{*} = {ɛ}_{m}^{′} - j \cdot ( {ɛ}_{m}^{″} + \frac{{σ}_{dc\_m}}{ω \cdot {ɛ}_{o}} ) = {ɛ}_{m}^{′} - j \cdot \frac{{σ}_{m}^{′}}{ω \cdot {ɛ}_{o}} \qquad ( 1 )$$

wherein ∈′ₘ is the real part and ∈″ₘ the frequency dependent imaginary part of the complex dielectric permittivity of the layer σ_{dc}_{_{—}}ₘ its static conductivity, σₘ its conductivity, ω the frequency of interest and ∈ₒ the vacuum permittivity.

The term “dielectric permittivity” as used here is understood to designate the relative permittivity of a material. It is generally a frequency dependent quantity. The dielectric properties described below are for simulations made in the frequency range of 15 MHz for comparison with measurements at a similar frequency.

When measuring glucose and many other properties of living skin and the underlying tissue, the most relevant layers of the tissue are, as shown in FIG. 3:

L1: The sebum. It has a typical thickness d₁=2 μm, a real dielectric permittivity ∈′₁ ranging between e.g. 3 (low water content, high fat content) and 80 (high water content), and a conductivity σ₁ in the order of 0.5-10⁻³ Sm/m depending on the physiological state (e.g. dry skin or skin with sweat).

L2: The stratum corneum. It has a typical thickness of d₂=10-20 μm, a real dielectric permittivity ∈′₂ of approximately 10 and conductivity in the order of 10⁻⁴-10⁻⁵ Sm/m.

L3: The epidermis having a typical thickness of d₃=100-300 μM, a real dielectric permittivity S′₃ of approximately 30 and a conductivity in the order of 0.02-0.04 Sm/m.

L4: The dermis having a typical thickness of d₄=500-1200 μm, a real dielectric permittivity ∈′₄ of approximately 110 and a conductivity in the order of 0.2-0.4 Sm/m.

L5: The fat having a typical thickness of d₅=1000-10000 μm, a real dielectric permittivity ∈′₅ of approximately 20 and a conductivity again in the order of 0.05-10⁻⁴ Sm/m.

Further layers below the fat, such as the muscle layer, have a limited interaction with the applied electric field from electrode arrangement 5 and can therefore be neglected, and the thickness of layer 5 can be set to ∞.

Also, some of the above layers, e.g. the thin sebum layer, may also be disregarded for simplified evaluations of the measurements.

The electrode arrangement applied to the surface 16 of the tissue is, in FIG. 3, depicted as consisting of a total of U electrode pairs u, with U=3. In this embodiment, each electrode pair forms one electrode configuration, to whose electrodes a voltage can be applied (while the other electrodes are e.g. in a high impedance state). A qualitative illustration of two field lines for each configuration is shown in FIG. 3.

It must be noted, though, that other electrode arrangements can be used as well, such as the one shown in FIG. 3 of WO 2005/120332.

In the first aspect of the present invention, the electrode arrangement may also consist of only a single configuration, i.e. U=1.

The present invention is based on the understanding that the dielectric permittivities of the various layers are affected differently by the property to be measured. For example, in the case of glucose, it is understood that a glucose variation gives rise to a strong variation of the dielectric permittivity of the dermis, while only weakly affecting the properties of the other layers. Hence, the purpose of the methods described in the following sections is to obtain the relevant parameters of individual layers.

The signals s measured by the device are generally a function of the effective complex capacitance C* of the electrode configuration that has been used, which, in turn, is a function of the effective dielectric permittivity of the tissue as seen by the electrode.

The complex capacitance C*, which is the inverse 1/Y* of the complex admittance Y*, can be written as

C*=C*_{f}+C*₀·∈_{eff}, (2)

where C*_{f} represents the complex capacitance of the base carrying the electrodes, C*₀ the additional capacitance in the absence of the tissue, and ∈_{eff} effective dielectric permittivity of the tissue as seen by an electrode configuration.

The device can be calibrated by determining C^{*}_{f} and C*₀. These parameters are determined by measuring C* for a number of reference liquids (in the place of the body tissue), at least two, but preferably a higher number, with known permittivity, and then by approximately solving (by linear regression) of the system of equations formed by the repeated application of eq. (2). In most cases, the system can be simplified by the assumption the air and the base are non-dispersive and the imaginary parts of C*_{f} and C*₀ are zero.

The effective dielectric coefficient ∈_{eff} can be expressed as a function Eᵤ of the dielectric coefficients ∈₁, . . . , ∈_{M} of the layers, their thicknesses, as well as the geometry of the electrode configuration u, i.e.

∈_{eff}=Eᵤ(∈₁, . . . , ∈_{M},d₁, . . . , d_{M-1},u). (3)

Depending on the complexity of the electrode configuration and the number M of layers, Eᵤ can be expressed either in closed, analytical form, or it has to be calculated numerically, see also below.

The measured signal s of the device can e.g. be ∈_{eff}, or it can be any parameter derived therefrom, such as capacitance C*, or amplitude A or phase shift φ as described above. Therefore, and in view of eq. (3), the measured signal s can be expressed as

s=F0ᵤ(∈₁, . . . , ∈_{M},d₁, . . . , d_{M-1}) (4)

with F0ᵤ being a function that describes the measured signal for given values ∈₁, . . . ∈_{M}, d₁, . . . d_{M-1} when using electrode configuration u.

In some parts of the following text, we only consider a single electrode configuration (U=1), and the thicknesses d₁ . . . d_{M-1} are assumed to be fixed, known values and are not of interest, while it is of importance that the dielectric coefficients ∈ₘ depend on frequency. In this case eq. (4) can be written as

s(ω)=F0(∈₁(ω),∈_{L}(ω)) (4a)

Processing of the measured signal s can proceed using one or both of the methods described in the following. The methods are termed “frequency analysis” and “spatial analysis”. The methods can be used individually or in combination.

Frequency Analysis:

This method is based on an analysis of the dispersion of the measured value s(ω) and on a model of the dispersion of the dielectric permittivity ∈ₘ of the layers 1 . . . M. For example, it is assumed that the dispersion of each layer can be described by the Havriliak-Negami relaxation, see e.g. S. Hevriliak and S. Negami, J. Pol. Sci.: Part C, 14, 99 (1966)

$${ɛ}_{m} ( ω ) = {ɛ}_{∞ , m} + \frac{{ɛ}_{0 , m} - {ɛ}_{∞ , m}}{{[ 1 + {( j \cdot ω \cdot {τ}_{m} )}^{{α}_{m}} ]}^{{β}_{m}}} , where 0 \leq α , β \leq 1 , \qquad ( 5 a )$$

with the parameters ∈_{∞,m}, αₘ, βₘ, τₘ, ∈_{0,m}. These parameters will, in general, be different for each layer. Some of the parameters may be known in advance, while others will depend on the property c to be measured or on some other state of the tissue that varies over time.

Another approach to model the dispersion of the dielectric permittivities is to use a mixture formula, where ∈ₘ(ω) is assumed to be generated by a mixture of components. The generalized formula is

$${( {ɛ}_{m} ( ω ) )}^{α} = {\sum }_{q = 1}^{Q} {{v}_{m , q} ( {ɛ}_{q} ( ω ) )}^{α} \qquad ( 5 b )$$

where v_{m,q} is the volume fraction of the q-th component of the mixture in layer m, ∈_{q}(ω) its frequency dependent complex dielectric permittivity and Q the number of components in the mixture. α is a parameter that changes from one model to another, with extreme values of 1 for parallel mixing and −1 for serial mixing.

As an example, the application to a skin layer can be implemented as follows. The skin layer is described by a two-component mixture of water and biological material. The dielectric permittivity of water is described in literature. The (dry) biological material has a permittivity in the range of 2.5 to 20 in the frequency range of evaluation. In the simplest example of a model, frequency independent permittivities are considered, however the frequency dependence can be added as an additional term for more complicated descriptions as indicated in Equation 5b. Equation (5b) is advantageously used with α=⅓ (Landau-Lifshitz-Looyenga's formula, see C. Böttcher, “Theory of Electric Polarization, Elsevier, Amsterdam, 1973, and reference therein, Landau Lifshitz, Electrodynamics of continuous media, Pergamon, Oxford 1960, and H. Looyenga, Physica 31 (1965) 401)) describing a mixture of two components, i.e. we have

∈m(ω)=(v_{m,1}·∈_{water}^{1/3}+(1−v_{m,1})·∈_{biol}^{1/3})³ (5c)

with v_{m,1} being the water content of layer m, ∈_{biol}=2.5, and ∈_{water} being given by

$${ɛ}_{water} = {ɛ}_{a ∞} + \frac{{ɛ}_{a 1} - {ɛ}_{a ∞}}{1 + ( {iωτ}_{a 1} )} - \frac{{iσ}_{dc}}{ω \cdot {ɛ}_{a 0}} \qquad ( 5 d )$$

with the single Debye dispersion parameters given by ∈_{a∞}=5.2, ∈ₐ₁=78.36, τₐ₁=8.27E-12 s, ∈ₐ₀=8.85E-12, ω being the angular frequency, σ_{dc} the direct current conductivity (in our case the one of serum, i.e. 1.4 S/m, see CRC Handbook of chemistry and physics (pure water at 25° C.).

The models of equations (5a)-(5d) are only a few of the various dispersion models that can be used for the present invention. Other models include e.g. additional Debye relaxations, Cole-Cole relaxation and Cole-Davidson relaxation that represent the special cases of eq. (5a) where α=1,β=1, α≠01, β=1 and α=1,β≠1 respectively, see e.g. K. S. Cole and R. H. Cole, J. Chem. Phys. 9, 341 (1941) and D. W. Davidson and R. H. Cole, J. Chem. Phys. 19, 1484 (1951).

Therefore, we can generalize eq. (3) to a generalized dispersion function H as follows:

∈ₘ(ω)=H(Pₘ₁, . . . , P_{mN},ω)) (6)

with m=1 . . . M and with dispersion parameters pₘₙ with m=1 M with M>1 and n=1 . . . N. In the example of eq. (3) we have ∈_{∞},m=Pₘ₁, αₘ=pₘ₂, βₘ=pₘ₃, τₘ=pₘ₄, ∈_{0,m}=p_{m5, and N=}5.

Combining (4a) and (6), the measured signal s can be expressed by a further function F1 as

s(ω)=F1(p₁₁, . . . , p_{MN},ω) (7)

(Here, we assume that all the measurements are carried out at the same electrode configuration, i.e. U=1, and that the thicknesses of the individual layers are known, fixed values, e.g. chosen on physiological observations, for which reason we use the formulation of eq. (4a) instead of (4).)

When Fl is known, e.g. from numerical simulations of eq. (4), eq. (7) can be calculated.

To determine the parameters pₘₙ, or at least part thereof (assuming that some of these parameters are known or remain permittivity over longer periods), we can proceed as follows:

1) The electrode arrangement is applied to the skin of the tissue, as shown in FIG. 3.

2) A plurality of electrical fields in the tissue are generated at different frequencies ω_{w} with w=1 to W, e.g. in the range between 100 kHz and 500 MHz in subsequent measurement.

3) For each frequency ω_{w}, the signal s_{w}=s(ω_{w}) is measured, thereby generating a measured dataset {(s₁, ω₁), (s_{W}, ω_{W})}.

4) Function F1 of eq. (7) is fitted to the measured dataset by varying at least part of the dispersion parameters pₘₙ.

In a simple approach, the parameters pₘₙ can be obtained from a conventional least-squares fitting algorithm that varies the parameters pₘₙ in order to find a best match of equations (7) to the calibration measurements. Suitable algorithms are known to a person skilled in the art and are e.g. described by Press, Teukolsky, Vetterling and Flannery in “Numerical Recipes in C”, Cambridge University Press, 2^{nd} edition, 1992, Chapter 15.

As mentioned, function F0 of equation (4a) can be obtained by various means. One example, based on a numerical analysis of the system, is given in the following.

First, it is assumed that function F0 can be expressed by a model function L having T model parameters r₁, . . . , r_{T}, i.e. we write, instead of (4a),

s=L(r₁, . . . , r_{T},∈₁, . . . , ∈ₘ) (8)

For example, for a two-layer model (M=2) and the assumption of a linear relationship, we have

s=L(r₁·r_{T},∈₁·∈_{M}) (9)

In order to determine the model parameter rₜ, an approximate solution as follows can be used:

1) A number K of numerical simulation steps are carried out, numbered k=1 . . . K. For each simulation step k, it is assumed that the dielectric permittivities of the layers have a certain set of values ∈₁ₖ, ∈_{Mk}. Starting from these values, a numerical approximation of the effective dielectric permittivity ∈_{eff} as seen by the electrode arrangement is calculated using e.g. the commercially available AC/DC simulation module of COMSOL Inc. (www.comsol.com), which is part of the COMSOL Multiphysics Simulation Platform. From the simulated value of the effective dielectric permittivity ∈_{eff} the corresponding value sₖ is calculated that would be measured for the given set of values. The result of each simulation step is represented as a vector vₖ=(∈₁ₖ, . . . , ∈_{Mk},Sₖ) with k=1 . . . K.

2) Model function L of eq. (8) is now fitted to match the vectors vₖ by varying the model parameters r₁, . . . r_{T}.

After this procedure, the model parameters r₁, . . . r_{T} are known, which allows to evaluate eq. (8) and therefore eq. (7) quickly.

### EXAMPLE

The tissue is modeled by a two-layer system, i.e. M=2, where the contributions of the sebum, stratum corneum and epidermis are assumed to originate from a first layer of e.g. a thickness d₁ of a fixed value between 100 μm and 200 μm, in particular 150 μm, while the second layer is the dermis layer where we assume that d₂=∞ is a necessary assumption for a two-layer system where the electrode arrangement is such that a deepest field does not extend beyond the dermis. The value of d₁ can e.g. be a fixed, predetermined value, or an individual, fixed value for each user.

In the simulation steps, the real and imaginary parts of the complex dielectric permittivities are each e.g. varied in 5 steps, which results in 5⁴=625 simulation steps. For example, the dielectric permittivities are varied in logarithmic steps between the following values:

∈′₁ from 1 to 700

∈′₂ from 50 to 300

σ′₁ from 0.05 to 1 Sm/m

σ′₂ from 0.001 to 0.4 Sm/m

Once the model parameters rₖ are known, eq. (8) and therefore eq. (7) can be calculated quickly when fitting eq. (7) to the data obtained in a measurement. The parameters pₘₙ obtained from this fitting process can then be used to determine the desired property c.

The exact procedure for determining c from the parameters pₘₙ depends on the nature of property c.

For example, as mentioned, if property c is the glucose concentration, the parameters pₘ₁ . . . p_{mN} of the dermis layer are the parameters most relevant for the determination of c.

Suitable methods for determining the glucose level from measured tissue parameters and calibration data are described in WO 2005/053526, the disclosure of which is incorporated herein by reference, in particular the section “Calibration” thereof.

Spatial Analysis:

This second method is based on an analysis of the response of the tissue to several applied electrical fields having different spatial distributions. To generate such fields, a plurality of different electrode configurations u=1 . . . U are used, i.e. U>1. A voltage is applied (subsequently) to each configuration u, so that differently distributed voltage patterns are applied to the investigated skin region. Typically, the voltage will be an AC-voltage having a frequency between 100 kHz to some GHz, and the frequency can be the same for all configurations, albeit different frequencies for different configurations can be used as well.

It has been shown (see Alanen, E. Lahtinen T. and Nuutinen J. IEEE Trans. Biomed. Eng, 45, no 10, 1241-1248 (1989)) that the penetration of the EM field depends on the characteristic geometry of the electrodes and the frequency of the applied electric field. This has been verified with in vitro measurements and finite element simulations of a 2 layers system with materials of known properties. FIG. 4 is an example illustrating this, where the first material is water with different salt concentration and the second layer is Teflon. The electrode with smallest geometry (white bars) measures mainly the dielectric properties (here the conductivity) of the first layer (dashed line), and with increasing electrode size (grey, then black), the measured properties approach the dielectric properties of the second layer (dotted line). This has been confirmed for different salt concentrations in the first layer, and for each concentration a comparison is shown between the measured (left column) and simulated (right column) values.

For each electrode configuration u, a corresponding value sᵤ is measured, i.e. eq. (4) can be written as

sᵤ=F0ᵤ(∈₁, . . . ∈_{M},d₁, . . . d_{M-1})tm (10)

In this manner, a measured dataset of U values {s₁, . . . s_{U}} is obtained.

In a next step, the independent variables ∈₁, . . . ∈_{M}, d₁, . . . d_{M-1} are used as parameters in the set of equations given by eq. (10) (for u=1 to U), and an approximate solution of this set of equations is sought by varying at least some of these parameters. Suitable algorithms are known to the person skilled in the art and e.g. described in the already mentioned textbook of Press, Teukolsky, Vetterling and Flannery in “Numerical Recipes in C”, Cambridge University Press.

Obviously, the number of (real-valued) equations in (10) should be larger than the (real-valued) degree of freedom of the parameters that are varied, taking into account that each of the equations in (10) is complex, i.e. 2·U real-valued equations are available if the number of measured configurations is U.

This procedure allows to determine the complex dielectric parameters ∈₁, . . . ∈_{M} and/or thicknesses d₁, . . . d_{M-1} of some or all of the layers of the tissue. These parameters or part of them (such as the dielectric permittivity of the dermis) can then be used for determining the glucose level or some other property of the tissue.

Solving the set of equations (10) requires the functions F0ᵤ to be calculated repetitively. Advantageously, the functions F0ᵤ are predetermined, i.e. they are determined prior to solving the equations (10). A method for predetermining F0ᵤ is described with reference to eqs. (8) and (9) above.

Another method for solving the set of equations (10) is based on reformulating these equations by moving the unknown, desired parameters ∈₁, . . . ∈_{M} and d₁, . . . d_{M-1} to the left-hand side, expressing them as functions of the measured values s₁, . . . s_{U}. The re-formulated set of equations looks as follows:

∈ₘ=G1ₘ(S₁, . . . s_{U}),for m=1 to M, (11a)

dₘ=G2ₘ(s₁, . . . s_{U}), for m=1 to M -1. (11b)

G1ₘ and G2ₘ are functions that can be determined prior to analyzing a specific set of experimental data. For example, G1ₘ and G2ₘ can be predetermined by numerically analyzing the system. For this purpose, similar to the procedure explained in reference to eqs. (8) and (9), the system is numerically analyzed, e.g. using the AC/DC Simulations Module by COMSOL Inc. as mentioned above, by calculating the measured values sᵢ, . . . s_{U} as a function of a given set of parameters cm and d₁, . . . d_{M-1}. This is repeated for a large number K of sets of simulation steps by varying the parameters within physiologically reasonable boundaries, wherein the result of each set can be represented by a vector vₖ=(∈₁ₖ, . . . ∈_{Mk}, d₁ₖ, d_{M-1,k}, S₁ₖ, . . . S_{Uk}) with k=1 . . . K. Similar as in eq. (8), model functions for eq. (11a), (11b) can then be set up, which model functions contain parameters r₁, . . . r_{T} and return the values of ∈ₘ and dₘ. These parameters can be determined by fitting the model functions to the data in the vectors vₖ.

In an alternative embodiment, the functions G1_{m and G2}_{m can be determined by mathematical analysis of a system having known electrode geometries and M layers.}

### Example A

Spatial analysis has been used for determining the glucose level in a plurality of experiments on human volunteers, comparing the thus obtained results with the glucose level measured by conventional, invasive techniques.

A simple two-layer system (M=2) was assumed, and the thickness of the first layer was assumed to be known and kept fixed at d₁=150 μm, i.e. it was assumed that the properties of the first three layers of FIG. 3 could be modeled with sufficient accuracy by a single layer.

σ′ was assumed to be 0 for all layers (which may be a poor assumption and will have to be replaced by more realistic values, e.g. as given above, in a more refined analysis).

The measurement was carried out with two electrode configurations, i.e. U=2. Configuration 1 was formed by a first pair of electrodes having a first mutual distance D1 and arrangement 2 was formed by a second pair of electrodes having a second mutual distance D2. The measured signals s₁ and s₂ were the real-valued capacitances Cₛₕₒᵣₜ and C_{1ong} measured for the two configurations.

The results of the finite element simulation were used for determining G1₁ and G1₂ of eq. (11a) (since the thickness d₁ was kept fixed, there was no need to determine G2₁ of eq. (11b)). Since the imaginary parts of the dielectric permittivities were assumed to be zero, the functions G1₁ and G2₂ become real-valued and were modeled as follows:

∈′₁=G1₁(s₁,s₂)=a₁+a₂C_{1ong}+a₃Cₛₕₒᵣₜ+a₄C_{1ong}Cₛₕₒᵣₜ (12ₐ)

∈′₂=b₁+b₂C_{1ong}+b₃Cₛₕₒᵣₜ+b₄C_{1ong}Cₛₕₒᵣₜ (12b)

with real-valued parameters a₁, . . . a₄ and b₁, . . . b₄, which can be determined using finite element analysis and subsequent least-squares fitting as described above.

The models of eq. (12a, 12b) were found to match the results well in a range of C_{1ong}=2.6·10⁻¹²−6.9·10⁻¹² Farad and Cₛₕₒᵣₜ=1.1·10⁻¹²−3.7·10⁻¹² Farad.

Glucose level c was estimated to be a function of the dielectric permittivities ∈₁ and ∈₂ as follows:

c=c₀+c₁·∈′₁+c₂∈′₁·∈′₂. (13)

The parameters c₀ and c₁ were determined by comparing invasively measured glucose levels to values of ∈₂ obtained by eq. (12b).

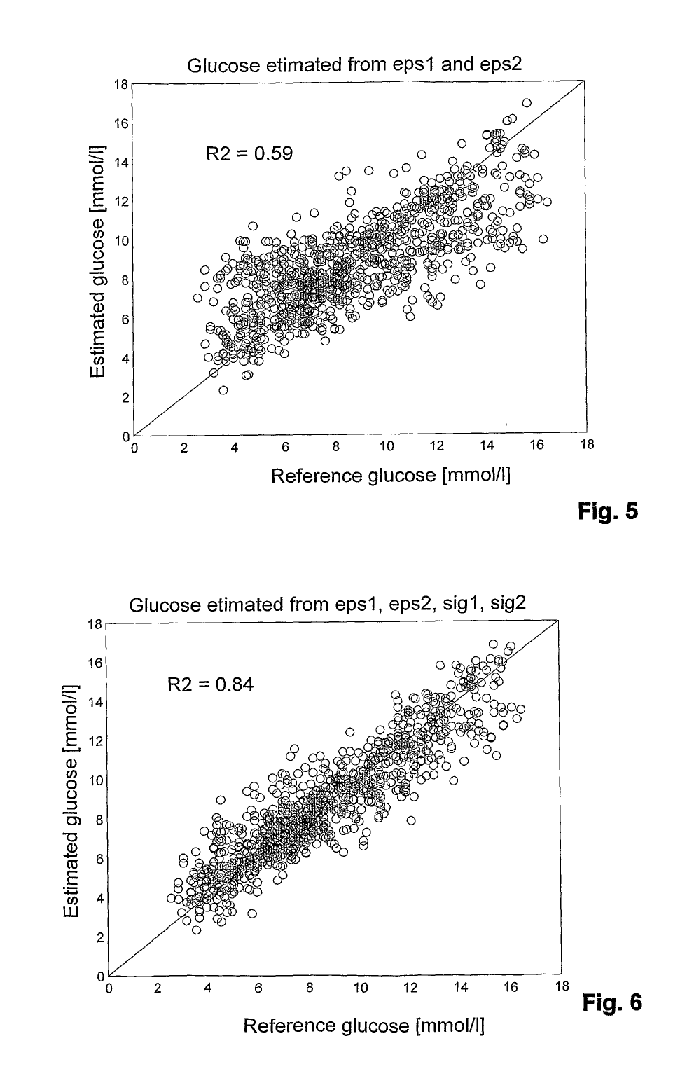

FIG. 5 shows a plot of the glucose level obtained by eq. (13) (vertical axis) vs. the glucose level measured by conventional, invasive technique (horizontal axis) for a series of experiments on human volunteers.

### Example B

For Eqs. (12a, 12b) the imaginary parts of the permittivities were assumed to be zero. In a refined model, non-zero imaginary permittivities are allowed for and expressed by non-zero conductivites σ₁, σ₂ for layer 1 and 2 as follows:

∈′₁=a₁+a₂C_{1ong}+a₃Cₛₕₒᵣₜ+a₄G_{1ong}+a₅Gₛₕₒᵣₜ+a₆C_{1ong}Cₛₕₒᵣₜ+a₇G_{1ong}Gₛₕₒᵣₜ+a₈C_{1ong}CₛₕₒᵣₜG_{1ong}Gₛₕₒᵣₜ (14a)

∈′₂=b₁+b₂C_{1ong}+b₃Cₛₕₒᵣₜ+b₄G_{1ong}+b₅Gₛₕₒᵣₜ+b₆C_{1ong}Cₛₕₒᵣₜ+b₇G_{1ong}Gₛₕₒᵣₜ+b₈C_{1ong}CₛₕₒᵣₜG_{1ong}Gₛₕₒᵣₜ (14b)

σ₁=D₁+D₂C_{1ong}+D₃Cₛₕₒᵣₜ+D₄G_{1ong}+D₅Gₛₕₒᵣₜ+D₆C_{1ong}CₛₕₒᵣₜD₇G_{1ong}Gₛₕₒᵣₜ+D₈C_{1ong}CₛₕₒᵣₜG_{1ong}Gₛₕₒᵣₜ (14c)

σ₂=E₁+E₂C_{1ong}+E₃Cₛₕₒᵣₜ+E₄G_{1ong}+E₅Gₛₕₒᵣₜ+E₆C_{1ong}Cₛₕₒᵣₜ+E₇G_{1ong}Gₛₕₒᵣₜ+E₈C_{1ong}CₛₕₒᵣₜG_{1ong}Gₛₕₒᵣₜ (14d)

where Gₛₕₒᵣₜ and G_{1ong} are the resistances measured for the two electrode configurations.

Glucose level c was estimated to be a function of the dielectric permittivities ∈₁ and ∈₂ and conductivities σ₁, σ₂ as follows:

c=c₀+∈′₁+c₂∈′₂+c₃σ₁+c₄σ₂ (15)

FIG. 6 shows a plot of the glucose level obtained by eq. (15) (vertical axis) vs. the glucose level measured by conventional, invasive technique (horizontal axis) for the experimental series as used in FIG. 5.

### Example C

In example B, no cross-terms of the conductances and permittivities were added. The results can be further improved by adding such cross-terms:

c=c₀+∈′₁+c₂∈′₂+c₃σ₁+c₄σ₂+c₅∈′₁²+c₆σ₁σ₂+c₇∈′₁²σ₁σ₂ (16)

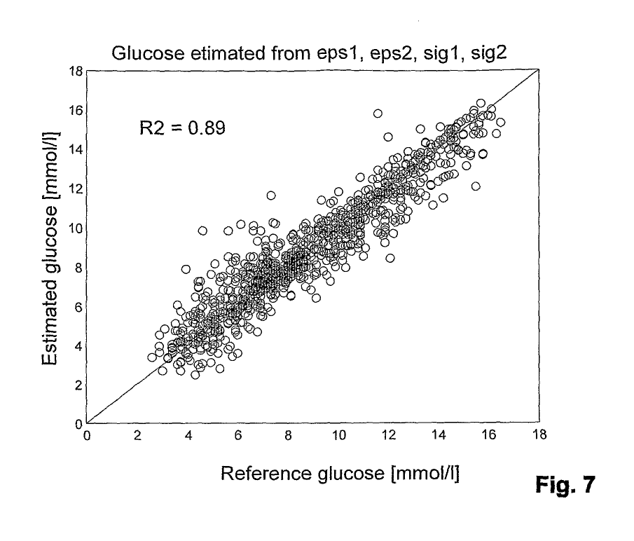

FIG. 7 shows the corresponding plot of the glucose level obtained by eq. (16) (vertical axis) vs. the glucose level measured by conventional, invasive technique (horizontal axis) for the experimental series as used in FIG. 5.

While there are shown and described presently preferred embodiments of the invention, it is to be distinctly understood that the invention is not limited thereto but may be otherwise variously embodied and practiced within the scope of the following claims.

### SYMBOLS

A: amplitude of the impedance
A₁, . . . , A₄: coefficients in model (13a)
a₁, . . . , a₈: coefficients in models (12a) and (14a)
B₁, . . . , B₄: coefficients in model (13b)
b₁, . . . , b₈: coefficients in model (12b) and (14b)
c: property to be determined, glucose level
C*: complex capacitance
C*_{f}. complex base capacitance
C*₀: complex air capacitance
Cₛₕₒᵣₜ, C_{1ong}: capacitance for short and long electrode configuration
c₀, . . . , c₈: parameters in model of eq. (14), (15) and (16)
D₁ . . . D₈: coefficients in model (14c)
dₘ: thickness of layer m
E₁ . . . E₈: coefficients in model (14d)
Eᵤ: function describing ∈_{eff} depending on ∈ₘ and dₘ for configuration u
F0ᵤ: function describing s depending on the dielectric permittivity and thicknesses of the layers of the electrode configuration u

F0: function describing s depending on the dielectric permittivities of the layers
F1: function describing s depending on pₘₙ and ω
G1ₘ: function describing ∈′ₘ as a function of the signals sᵤ measured for all electrode configurations u=1 to U
G2ₘ: function describing ∈″ₘ as a function of the signals sᵤ measured for all electrode configurations u=1 to U
G3ₘ: function describing dₘ as a function of the signals sᵤ measured for all electrode configurations u=1 to U
H: dispersion function
k: index of vector v
K: number of vectors vₖ
L: model function describing what signal is measured for given dielectric permittivities of the layers
m: index for layers
M: number of layers
n: index for dispersion parameter
N: number of dispersion parameters
pₘₙ: dispersion parameter with index n for layer m
q: index for components in mixture
Q: number of components in mixture
s: measured signal
sₖ: signal measured for the dielectric permittivities in vector vₖ
sᵤ: signal measured for electrode configuration u
sᵤₖ: signal measured for the dielectric permittivities and thicknesses in vector vₖ for electrode configuration u
s_{uw},: signal measured at frequency having index w
t: index of model parameter for model function L
T: number of model parameters in modem function L
u: index for electrode configurations
U: number of electrode configurations
v_{q}: volume fraction of component q in mixture
w: index for frequencies
W: number of frequencies
αₘ: dispersion parameter α for layer m
βₘ: dispersion parameter β for layer m
γₘ: dispersion parameter γ for layer m
∈: complex dielectric permittivity
∈′: real part of dielectric permittivity
∈″: imaginary part of dielectric permittivity
∈_{eff}: effective dielectric permittivity of the tissue as seen by an electrode configuration
∈ₘ: complex dielectric permittivity of layer m
∈ₘk: complex dielectric permittivity of layer m in vector k
∈_{q} complex dielectric permittivity of component q in mixture
∈ᵤ: complex dielectric permittivity as seen for electrode configuration u
∈_{0,m}: dispersion parameter ∈₀ for layer m
∈_{∞,m}: dispersion parameter ∈_{∞} for layer m
∈ₘₖ: complex dielectric permittivity of layer m in vector vₖ
∈ₐ₀: dispersion parameter for water
∈_{a∞},: dispersion parameter for water
∈ₐ₁: dispersion parameter for water
∈ₐ₂: dispersion parameter for water
τₐ₁: dispersion parameter for water
τₐ₂: dispersion parameter for water
φ: impedance phase shift
σ_{s,m}: parameter σₛ for layer m
σ_{dc}: dispersion parameter for water
σ₁,σ₂:conductivity of layers 1 and 2
τₘ: dispersion parameter τ for layer m
ω: frequency
ω_{w}: frequency at index w

1. A method for measuring a property c of living tissue, which property c affects the complex dielectric permittivity ∈(ω) of said tissue, comprising the steps of
applying an electrode arrangement to a skin region of said tissue,
generating, by means of said electrode arrangement, a plurality of electrical fields in said tissue at different frequencies ω_{w} with w=1 to W and measuring, for each of said frequencies, a signal s_{w} with w=1 to W, depending on the dielectric permittivity ∈(ω_{w}) as seen by said electrode arrangement at the frequency ω_{w}, thereby generating a measured dataset {(s₁, ω₁), . . . (s_{W}, ω_{W})},
using dispersion parameters pₘₙ with m=1 M with M>1 and n=1 . . . N, wherein said dispersion parameters pₘₙ are parameters of a dispersion function H describing a dispersion of the dielectric permittivity ∈ₘ of a virtual homogeneous tissue layer m in said skin region by
∈ₘ(ω)=H(pₘ₁, . . . , p_{mN},ω),
with m=1 . . . M,
fitting a function F1
s(ω)=F1(p₁₁, . . . , p_{MN},ω)
to said measured dataset {(s₁, ω₁), . . . (s_{W}, ω_{W})} by varying at least part of said dispersion parameters pₘₙ, wherein said function F is given by
F1(p₁₁, . . . p_{WL},ω)=F0(∈₁(ω), . . . ∈_{M}(ω))
with a function F0(∈₁(ω), . . . ∈_{L}(ω)) describing the signal s(ω) measured if said layers 1 . . . M have the dielectric permittivities ∈₁(ω), . . . ∈_{M}(ω),
said method further comprising the step of using at least part of the varied dispersion parameters pₘₙ for calculating said property c.

2. The method of claim 1, further comprising the steps of deriving said function F0 by
obtaining a plurality of vectors vₖ=(∈₁ₖ, . . . ∈_{Mk}, sₖ) with k=1 . . . K, wherein each vector vₖ comprises the signal sₖ that would be measured at said electrode arrangement if said layers had the dielectric permittivities ∈_{Mk},
fitting a model function L
s=L(r₁, . . . r_{T}, . . . ∈_{Mk})
to said vectors vₖ by varying model parameters r₁, . . . r_{T} of said model function L and
using the varied model parameters r₁, . . . r_{T} for calculating
F0(∈₁(ω), . . . ∈_{L}(ω))=L(r₁, . . . r_{T},∈₁(ω),∈_{L}(ω)).

3. The method of claim 2, wherein said model function L is linear in r₁, . . . r_{T}.

4. The method of claim 1, wherein
$$\begin{matrix}{ɛ}_{m} ( ω ) = H ( {p}_{m 1} , … , {p}_{mN} , ω ) \\ = {ɛ}_{∞ , m} + \frac{{ɛ}_{0 , m} - {ɛ}_{∞ , m}}{{[ 1 + {( j \cdot ω \cdot {τ}_{m} )}^{{α}_{m}} ]}^{{β}_{m}}} ,\end{matrix}$$
$$where 0 \leq α , β \leq 1$$
with ∈_{∞,m}=pₘ₁, αₘ=pₘ₂, βₘ=pₘ₃, τₘ=pₘ₄, ∈_{0,m}=pₘ₅, and N=5.

5. The method of claim 1, wherein
$$\begin{matrix}{ɛ}_{m} ( ω ) = H ( {p}_{m 1} , … , {p}_{mN} , ω ) \\ = {( {ɛ}_{m} ( ω ) )}^{α} \\ = {\sum }_{q = 1}^{Q} {{v}_{m , q} ( {ɛ}_{q} )}^{α}\end{matrix}$$
where v_{m,q} is a volume fraction of the q-th component of a mixture in layer m, ∈_{q} the complex dielectric permittivity of the q-th component and Q the number of components in the mixture, ∈_{q} is ∈_{q}(ω) for at least some values of q and α a number between −1 and 1.

6. The method of claim 1, wherein M=2.

7. The method of any claim 1, wherein a thickness of a topmost layer of said skin area is between 10 and 300 μm.

8. A method for measuring a property c of living tissue, which property c affects the complex dielectric permittivity c of said tissue, comprising the steps of:
applying an electrode arrangement to a skin region of said tissue,
generating, by means of said electrode arrangement, a plurality of electrical fields in said tissue, by applying voltages to different configurations u with u=1 to U and U>1 of said electrode arrangement, and measuring, for each of said configurations, a signal sᵤ with u=1 to U, depending on the dielectric permittivity ∈ᵤ as seen by said electrode arrangement for configuration u, thereby generating a measured dataset {s₁, . . . s_{U}},
using a set of dielectric parameters ∈₁, . . . ∈_{M} and thickness parameters d₁, . . . d_{M-1} describing the dielectric permittivity and thickness of a set of M homogeneous tissue layers in said skin region,
solving a set of equations
sᵤ=F0ᵤ(∈₁, . . . ∈_{M},d₁, . . . d_{M-1}),
with u=1 to U, by varying at least part of said complex dielectric parameters ∈₁, . . . ∈m and/or said thickness parameters d₁, . . . d_{M-1}, wherein said function F0ᵤ describes the signal sᵤ measured if said layers 1 . . . M have the complex dielectric parameters ∈₁, . . . ∈m and thickness parameters d₁, . . . d_{M-1} and if the configuration u is used,
said method further comprising the step of using at least part of the varied real and imaginary dielectric parameters ∈₁, . . . ∈_{M} and/or at least part of the thickness parameters d₁, . . . d_{M-1} for calculating said property c.

9. The method of claim 8, wherein at least part of said voltages applied to the different configurations u have equal frequency but are applied by applying differently distributed voltage patterns to said skin region.

10. The method claim 8, wherein said set of equations is solved by using predetermined functions F0ᵤ.

11. The method of claim 8 wherein said set of equations is solved by using a predetermined set of functions G1ₘ and G2ₘ describing the real and imaginary dielectric parameters ∈′₁, . . . ∈′_{M}, σ′₁, . . . σ′_{M} and/or said thickness parameters d₁, . . . d_{M-1} as a function of said signals sᵤ as
∈ₘ=G1ₘ(s₁, . . . s_{U}),for m=1 to M,
dₘ=G2ₘ(s₁, . . . s_{U}), for m=1 to M−1.

12. A device for measuring a property c of living tissue, in particular a glucose level, which device comprises a control unit adapted to carry out the steps of claim 1.

13. The device of claim 12 further comprising:
an electrode arrangement,
a signal source controlled by said control unit and generating an electrical signal to be applied to said electrode arrangement for generating an electrical field in said tissue, and
a detector for measuring a response from said tissue to said electrical field and for determining the at least one property therefrom.

14. A method as claimed in claim 1, wherein said property of a living tissue is a glucose level.

15. A method as claimed in claim 8, wherein said property of a living tissue is a glucose level.
